(19) United States
(12) Patent Application Publication
(10) Pub. No.: US 2013/0317058 A1
(43) Pub. Date: Nov. 28, 2013

(54) OPHTHALMIC COMPOSITION FOR ENABLING DILATION OF PUPILS

(71) Applicant: Krishnasamy Rengasamy, Chennai (IN)

(72) Inventor: Krishnasamy Rengasamy, Chennai (IN)

(73) Assignee: M/S. APPASAMY ASSOCIATES, Chennai (IN)

(21) Appl. No.: 13/985,722

(22) PCT Filed: Feb. 17, 2012
(86) PCT No.: PCT/IN2012/000114

§ 371 Date: Aug. 20, 2013

(30) Foreign Application Priority Data
Feb. 18, 2011 (IN) 170/CHE/2011

(51) Int. Cl. A61K 31/439; A61K 31/167; A61K 31/216

(52) U.S. Cl. CPC A61K 31/439; A61K 31/216; A61K 31/167
USPC 514/304

(57) ABSTRACT

The present invention discloses an ophthalmic composition for ophthalmic application(s) inclusive of Eye examination, surgeries carried out in the posterior chamber of the Eye like cataract surgery. The said composition comprises at least three active ingredients. The said ingredients may be inclusive of, but not restricted to, Epinephrine Bitartrate, Atropine Bisulphate, Xylocaine Hydrochloride and additives like, antioxidant compounds, buffers and the like. The said composition comes in a packaging container inclusive of sterile package such as vial/ampoule/prefilled syringes. The said composition can be directly injected in the Eye in order to bring about effective and speedy dilation of the pupil(s), in particular pupil(s) of relatively smaller dimension than normal pupil. The three active ingredients are mixed in a predefined percentage range in a predefined volume and the composition is supplied in a sterile package.

### FIELD OF THE INVENTION

The present invention relates to, an ophthalmic composition. More particularly, the present invention relates to an ophthalmic composition, to enable dilation of Pupil(s), during ophthalmic application(s), inclusive of Eye examination and for increasing the overall efficacy of surgeries carried out in the posterior chamber of the Eye.

The ophthalmic composition as exemplified by the present invention is especially useful, when it is deployed for Eye examination and surgeries, that are performed in the posterior chamber, exemplified by Cataract surgery, Glaucoma, Retinal surgery, Vitreous Surgery and Implant of other devices in Posterior Chamber of the Eye, wherein, it is required to widen the dimension of the Iris by dilating the pupil(s) so that, the surgery can be performed efficiently.

Still further, the composition as exemplified by the present invention is especially useful when it is deployed in the patient's whose pupils do not respond to the conventional ophthalmic composition and take a relatively longer time to dilate, said pupils being termed as “sluggish pupils” by persons skilled in the art. The ophthalmic composition as exemplified in the present invention enables even the sluggish pupils to dilate in a quickened manner compared to conventional compositions.

In this context, structurally, the Eye comprises Anterior and Posterior chambers. Behind the iris (I), is a cavity called the anterior chamber (AC), filled with fluid, and behind that, is the lens (L). The anterior chamber (AC) is the fluid-filled space inside the Eye between the iris and the cornea's innermost surface, the endothelium. Behind the lens (L) is a second fluid-filled area called the posterior chamber (PC). It makes up most of the Eye's (E) entire size. The retina (R) is the surface that borders it in a large curve extending almost to the Ciliary Muscle around the lens. Retinal cells are highly light-sensitive and receive the images in incoming light.

### BACKGROUND OF THE INVENTION

It is known that, in the pre-industrial era, manufacturing of products was performed by manual operation, or by mechanical means. The economy was also predominantly restricted to territories such as, the village and/or town of inhabitation. The nutrition consumed by people, was also natural, and without artificial additives. However, with the advent of industrial era, the scenario of day-to-day living and the general lifestyle of people have undergone a transformation.

Environmental pollution has increased due to the establishment of factories, and industrial entities. The modes of nutrition have also changed with people consuming foods with processed ingredients as against natural ingredients in the past.

The changing lifestyle and consumption of poor nutrients, has caused a plethora of health problems. In particular, it has been observed that, due to the environmental pollution, Eye-related disorders have been on the rise. Some of the Eye-related disorders that have been observed are cataract, glaucoma and the like.

In the above-mentioned context, it has been further observed that, although, some of the Eye-related disorders are curable by instrumentation, such as, spectacles and contact lenses, others require medication to be used.

At an advanced stage, some of the disorders are curable only by Eye surgery. With particular focus to cataract, in medical terminology, a “cataract” is defined as opacity of the lens of the Eye that causes a progressive, painless loss of vision. The symptoms through which, cataract is recognizable are, in general, vision may be blurred, contrast may be lost, and halos may be visible around lights.

In this context, it is known that, Cataracts are the leading cause of blindness worldwide. Cataracts are common, mostly in the older adults. They usually develop without any apparent cause. Even though, Cataracts usually cannot be attributed to any apparent cause, however, certain factors have been identified as contributing factors. The identified contributing factors are inclusive of, but are not restricted to, injury to the Eye, prolonged use of certain drugs (such as corticosteroids), prolonged exposure to x-rays (such as with radiation therapy to the Eye), inflammatory and infectious Eye diseases, diseases such as diabetes, dark Eyes, prolonged exposure to direct sunlight, poor nutrition, smoking, alcohol use and heat from infrared exposure.

In the medical field, it is generally observed by Eye surgeons and the knowledgeable optical professionals that, people who have had a cataract in one Eye are more likely to develop one later in the other Eye. Sometimes, cataracts can develop in both Eyes at the same time. Babies can be born with them (congenital cataracts), and children can also develop cataracts, usually as a result of injury or illness. An ophthalmic professional is generally aware of the symptoms that may confirm the existence of a cataract.

In this context, because all light entering the Eye passes through the lens, clouding of the lens can block, distort, or diffuse light and cause poor vision. The first symptom of a cataract may be seeing halos and starbursts around lights (glare). Sometimes, the first symptom is blurred vision. Less commonly, double vision is an early symptom. A person may also notice that colors seem more yellow and less vibrant. Reading may become more difficult because of a worsening ability to distinguish the contrast between the light and dark of printed letters on a page. The extent of variation in vision due to the formation of cataract depends on the intensity of light entering the Eye and on the location of the cataract.

With a cataract near the back of the lens (posterior sub capsular cataract), visual acuity is worse when the pupil constricts (for example, in bright light or during reading). Posterior sub capsular cataracts are also more likely to cause loss of contrast, as well as glare from bright lights or car headlights, while driving at night. People with cataracts who take drugs that constrict their pupils, (certain glaucoma Eye drops, for example) may also have greater vision loss.

After the condition of cataract is determined, a doctor can usually detect a cataract, while examining the Eye with an ophthalmoscope. A doctor can identify the exact location of the cataract and the extent to which it blocks light by using an instrument called a slit lamp, which allows examination of the lens and other parts of the Eye in more detail. The only treatment that provides a cure for cataracts is surgery. There are no Eye drops or drugs that will make cataracts go away. Occasionally, cataracts cause changes (such as swelling of the cataract or glaucoma) that lead doctors to recommend the cataract be removed quickly. However, most times people, should have surgery only when their vision is so impaired by cataracts that, they feel unsafe, uncomfortable, or unable to perform daily tasks. There is no advantage to having cataracts removed before then.

Cataract surgery can be performed on a person of any age and is generally safe even for people with illnesses such as heart disease and diabetes. Usually, the doctor makes a small incision in the Eye and removes the cataract by breaking it up with ultrasound and taking out the pieces (phaco-emulsification). When all the cataract pieces have been removed, the surgeon places an artificial lens (intraocular lens) within the lens capsule left behind, when the cataract was removed.

Cataract surgery has been mentioned only for the purposes of illustration and Eye surgery is inclusive of but not restricted to surgeries carried out in the Posterior Chamber of the Eye like. Glaucoma, Retinal surgery, Vitreous Surgery and Implant of other devices in Posterior Chamber of the Eye.

It is known to an ophthalmic professional, that Eye surgery is an intricate procedure, and requires a lot of meticulousness and care, when the surgery is under progress.

To examine the process in detail, it is amply clear to a person skilled in the art that cataract surgery involves removal of the natural lenses of the patient's Eye, followed by implantation of replacement lenses termed as Intraocular lenses (IOL's) which are implanted behind the iris. In order to achieve such an Intraocular lens transplant, the Eye surgeon typically dilates the Eye, installs instrumentation to maintain the iris in an expanded position, and makes an incision in the anterior wall of the capsule, so as to permit removal of the patient's natural lens. At this point, the IOL is inserted and properly emplaced, followed by necessary final sutures in the cornea.

As can be appreciated, surgery of this type involves working in extraordinarily close quarters with the Iris. The Iris widens in low light conditions and contracts in bright light conditions in the natural scheme of things without manual intervention. But during surgery due to the brightness of microscope light, it is found that the Iris contracts, but it can be adverse to the results of the surgery, since there is a requirement for dilation of pupils during the surgery. To achieve the said dilation of pupils, certain pupil dilatory instruments are available. The said instruments are termed as “Iris Retractors”.

For instance, U.S. Pat. No. 5,174,279 entitled “Iris retractor for use in operations on the Eye of a living creature” reveals an Iris retractor for use in operating on the Eye of a living creature, by means of which the iris is drawn outwards for widening the pupil and is held at the outer edge, which is essentially formed by the transition from the cornea to the sclera. The iris retractor essentially comprises a suspended part having a hook portion and a guide part and on which a platelet-like clamping part is displaceable relative to the hook portion.

Similarly, U.S. Pat. No. 4,782,820 entitled “Iris retaining device” reveals an improved, iris retaining device adapted for placement within the pupillary opening of a human Eye so as to engage the inner margin of the iris, so as to enlarge and maintain the pupillary opening during Eye surgery. The device preferably comprises an elongated, arcuate, flexible, resilient body including a generally U-shaped in cross section iris-receiving sidewall; the latter presents an inboard central bight portion and a pair of spaced apart wall sections extending outwardly therefrom. The ends of the body are slidably interengaged, and a drawstring is provided for manually contracting the body for initial placement thereof. After such contraction and initial placement, the drawstring and may be released so that the resilient body expands to engage, expand and protect the margin of the iris.

As indicated, the conventional technique is to employ iris-expanding instrumentation, but this is only partially effective. Due to the usage of the instrumentation, the iris may be readily traumatized during Eye surgery, resulting from irrigation solutions used to wash out the cortex of the Eye, and instrumentation necessary to retract the iris during surgery. Finally, the iris can also be damaged during implantation of an IOL.

Many a time these retractors are cumbersome to insert into small incisions and result in faulty positioning. As a result of their relatively small size, the surgeon has oftentimes great difficulty to see the individual iris retractors during the process of inserting the retractors into the anterior chamber and attaching the retractors to the iris or removing them from the Eye. Sometimes, this problem generally requires a surgeon to interrupt the surgery to re-grasp and retract the iris.

Alternatively, to save time, surgeons must simply pull the iris retractor. This latter method can be dangerous because of possible lacerations to the delicate iris. This may potentially adversely affect the surgery.

Alternatively there are devices being used in Eye surgery today which retract the iris, stretching for 360 degrees around the iris, thereby enlarging the pupil, but these retractors must be held by someone, such as a surgeon or a nurse. Furthermore, the iris retractors currently in use do not provide equal stretching for 360 degrees around the iris.

When the related procedures requiring dilation are finished, the reverse procedure is carried out. It is found that the Iris does not return back to its original profile because of the apertures made in the Iris to insert the Iris Retractor.

Therefore, it is observed that a chemical composition when used to dilate a pupil is much more safer to avoid injury to the iris, a critical but weak and delicate organ. In procedures passing through or near the iris, it is desirable to dilate the pupil to keep the iris out of harm's way and to give the surgeon a wider opening through which to work and view the posterior segments of the Eye

Compositions are available in the art, which is employed for treatment of Eye-related disorders.

A proposal in the art is available, namely, U.S. Pat. No. 6,218,428, entitled “Ophthalmic composition”. The said proposal reveals an ophthalmic composition that combines multiple agents necessary to perform a routine Eye examination. The said composition is a mixture of a topical local anesthetic, a pupillary dilating agent, and a dye, all in solution. The composition may also include a preservative, a wetting agent, a diluting agent, and/or a buffer. The preferred composition provides a means to: 1) anesthetize the cornea to allow for pain relief, manipulation and the measurement of intraocular pressure, 2) dye the cornea and conjunctiva to allow for the detection of pathology and for the measurement of intraocular pressure, and 3) dilate the pupil to allow for examination of the optic nerve, macula, retina, retinal vasculature, and peripheral fundus.

The typical dilating agent used to dilate the pupils for examination of the posterior pole are sympathomemetic or parasympatholytic agent, or both, including phenylephrine, tropicamide, cyclopentolate, hydroxyamphetamine, atropine, homatropine and/or other dilating agents. Most preferably, the total dilating agent should be in an amount between about 0.1% and about 5% of the solution. Also an embodiment of this composition may be used preoperatively before ophthalmic surgery (e.g., cataract surgery).

A proposal in the art is available, namely, U.S. Pat. No. 7,229,961, entitled “Compositions and methods for enhancing drug delivery across and into ocular tissues”. The said proposal reveals Sympathomimetic agents, which are useful for treating, e.g., glaucoma and mydriasis. Exemplary sympathomimetic agents include, e.g., dipivefrin, epinephrine, phenylephrine, apraclonidine, cocaine, hydroxyamphetamine, naphazoline, and tetrahydrozoline. Muscaric antagonists are useful for treating, e.g., cycloplegic retinoscopy and cycloplegia. They are also useful in dilated fundoscopic exams. Exemplary muscaric antagonists include, e.g., atropine, scopolamine, homatropine, cyclopentolate, and tropicamide.

Yet another proposal in the art is available, namely. U.S. Pat. No. 5,227,372 entitled “Method for retaining ophthalmological agents in ocular tissues”. The proposal reveals Ophthalmological agents which may be used in combination with glucan sulfate as a vehicle readily may be determined by the skilled artisan by a number of techniques and include: therapeutic agents for the treatment of glaucoma such as epinephrine or its salt, therapeutic agents for the treatment of cataracts such as pirenoxine; anti-allergic agents such as sodium nomoglicate, cromolyn and amlexanox; anesthetics such as xylocaine, tetracaine and the like.

Another proposal in the art is available, namely, U.S. Pat. No. 6,268,359, entitled “Preventives or remedies for vision disorders”. The said proposal reveals a pharmaceutical composition for preventing or treating visual functional disorders caused by ocular circulatory disorders (for example, ocular hypertension and glaucoma) and visual function disorders, based on ciliary tension, (for example, myopia) whose active component is a vasopressin antagonist. In addition to the vasopressin antagonist, the pharmaceutical composition of the present invention may optionally contain, or may be used together with one or more other pharmacological active agents. Examples of such pharmacological active agents include, parasympathomimetic drugs (for example, pilocarpine and carbachol), cholinesterase inhibitors (for example, physostigmine salicylate, distigmine bromide and echothiopate iodide), sympathomimetic drugs (for example, epinephrine, dipivalyl epinephrine, clonidine, paraamino clonidine and brimonidine), and sympathomimetic Beta.-Blockers (for example, betaxolol, levobunolol, timolol and carteolol), prostaglandine derivatives (for example, isopropyl unoprostone and latanoprost) for diseases caused by ocular circulatory disorders, and tropicamide for diseases caused by ciliary muscle tension.

Still further, another proposal in the art is available, namely, U.S. Pat. No. 5,079,253 entitled, “Composition for the topical treatment of glaucoma or ocular hypertension. The said proposal reveals a method for the topical treatment of glaucoma or ocular hypertension, which comprises contacting the surface of the Eye with a composition, consisting essentially of an effective intra-ocular pressure reducing amount of a mixture of: an adrenergic agonist selected from the group consisting of epinephrine, dipivalylepinephrine, norepinephrine, phenylephrine, clonidine, isoproterenol, salbutamol, metaproterenol and terbutaline, and; a phosphodiesterase inhibitor selected from the group consisting of isobutylmethylxanthine, theophyllamine, Rolipram and RO-2017624, in an ophthalmically compatible carrier.

Still further, another proposal in the art is available, namely, U.S. Pat. No. 4,190,642 entitled, “Ocular therapeutic system for dispensing a medication formulation”. The said proposal reveals an ocular system, which is disclosed for dispensing a medication formulation to the Eye. The system comprises a formulation, consisting of a pilo-carpine osmotic solute and an epinephrine osmotic solute, dispersed in a polymer such that, the formulation is surrounded substantially, individually by the polymer. The system, when placed in the Eye, dispenses the formulation at a controlled rate, over time. A method also, is disclosed for the management of intraocular pressure using the ocular system.

Some other similar proposals are available in the art. It is observed that, the said proposals available in the art, describe usage of a chemical composition or a mechanical instrumentation, both of which are deployed to dilate the pupil to the required dimension for the duration of the surgery. The available proposals have some inherent deficiencies, however, for an Eye surgeon performing an intricate Eye surgery, it is required to dilate the pupil of the Eye during the duration in which, the surgery is performed.

It will be well understood by a person skilled in the art that, the pupil(s) are dilated not only for the purpose of Eye surgery inclusive of Cataract surgery, Glaucoma, Retinal surgery, Vitreous Surgery and Implant of other devices in Posterior Chamber of the Eye but also for the examination of the Eye for purposes other than eye surgery.

There is therefore, a need in the art, for an alternative to an instrument illustratively embodied by an Iris Retractor deployed conventionally to dilate the Iris, and widening the pupil within the Iris for the purpose of Eye surgery and also for the examination of the Eye for purposes other than Eye surgery. It is further observed by persons skilled in the art that, dilating the pupil depends on the nature of the pupil and its responsiveness due to age and other factors. Also many at time certain pupils termed by ophthalmic professionals as “sluggish pupils” take a longer time to dilate than “normal pupils” when conventional ophthalmic compositions are used. In such a scenario there is a need in the art for an opthalmic composition which enables quickening the process of dilating even in the “sluggish pupils”.

There is therefore, also a further need in the art, for an Ophthalmic Composition, in which, it is possible to dilate the pupil(s) for the required duration, especially for people who have pupil(s) which are relatively smaller in dimension than the normal pupil(s), and then, reverse it to its normal dimension without any detrimental side effects to the structure of the Iris which is a critical component for vision but which is fragile due to which, any laceration in the Iris structure will result in long-term permanent vision impairment.

### SUMMARY OF THE INVENTION

The present invention is embodied in the ophthalmic composition in a manner, so as to fulfill the above-mentioned need(s) in the art. In addition, the present invention also, has further objective(s) as enumerated and described in the present disclosure. A person skilled in the art, is very well aware that, the enumerated objective(s) of the composition, as embodied in the present invention, are merely illustrative of the scope and purview of the present invention, as described in the present disclosure.

However, such an illustration may not be construed to restrict the present invention in any manner. It will be very clear to a person skilled in the art that, any variation and/or combination in the said objective(s) fulfilled by the present invention, may also be understood to be within the scope and purview of the present invention, as described in the present disclosure. The present invention may be embodied in other specific forms without departing from the spirit or essential attributes thereof.

In the above-mentioned context, an objective of the present invention is, to provide an ophthalmic composition, using which, it is possible to dilate the pupil(s) during ophthalmic application(s) inclusive of Eye examination and surgeries carried out in the posterior chamber of the Eye like cataract surgery.

An objective of the present invention is to dilate even sluggish pupil(s) and therefore, conventionally take a longer time duration to dilate in a quick manner, so as to make the process of surgery non-cumbersome and more comfortable to the patient.

An objective of the present invention is to dilate pupil(s) which are relatively smaller in dimension than the normal pupil(s).

Yet another objective of the present invention is, to provide an ophthalmic composition, using which, it is possible to dilate the pupil during ophthalmic application(s) inclusive of Eye examination and surgeries carried out in the posterior chamber of the Eye like cataract surgery, in a manner that the dilated pupil(s) are capable of being restored to its original dimensions without deformities, after the surgery has been completed.

Still another objective of the present invention is, to provide an ophthalmic composition, using which, it is possible to dilate the pupil during ophthalmic application(s) inclusive of Eye examination and surgeries carried out in the posterior chamber of the Eye like cataract surgery, in a manner so that, the composition is nontoxic to the Eye.

Further still, an objective of the present invention is to provide an ophthalmic composition, using which, it is possible to dilate the pupil during ophthalmic application(s) inclusive of eye examination and surgeries carried out in the posterior chamber of the Eye like cataract surgery, in a manner such that, the composition retains the pupil in the same dimension, during the duration of the surgery or examination.

A further objective of the present invention is, to provide an ophthalmic composition, using which, it is possible to dilate the pupil without affecting the position or condition of the surrounding parts of the Eye, the Eye being an intricate and delicate organ, wherein, any minor change in positioning and/or condition of the surrounding parts of the Eye is capable of affecting the outcome of the surgery.

A further objective of the present invention is, to provide an ophthalmic composition, using which, it is possible to dilate the pupil without causing any laceration to the pupil resulting in deformities due to which there may be long term and permanent vision impairment due to usage of pupil dilatory instrumentation.

Further still, an objective of the present invention is to provide an ophthalmic composition, using which, it is possible to dilate the pupil during Eye surgery, without the requirement of the person effecting the pupil dilatory composition/instrumentation to be a very highly skilled ophthalmic professional for instance a person in the league of an Eye surgeon.

Further still, an objective of the present invention is to provide an ophthalmic composition, in which, it is possible to dilate the pupil during ophthalmic application(s) inclusive of Eye examination and surgeries carried out in the posterior chamber of the Eye like cataract surgery, without the requirement of a very costly instrument, since Iris Retractors are very expensive in comparison to Compositions.

Further still, an objective of the present invention is to provide an ophthalmic composition, in which, the Iris returns to its original configuration without any ruptures in its structure resulting in deformities, after the surgery is completed.

In view of the above-mentioned objectives, the present invention discloses an ophthalmic composition for enabling dilation of pupil(s), in ophthalmic application(s) inclusive of Eye examination and surgeries carried out in the posterior chamber of the Eye like cataract surgery. The ophthalmic composition as embodied in the present invention comes in a packaging container inclusive of sterile package such as vial/ampoule/prefilled syringes. The ophthalmic composition as embodied by the present invention is an Intacameral Mydriatic solution (ICM) Injected for pupil dilation, during Eye examination and surgeries carried out in the posterior chamber of the Eye.

In medical parlance, an Intacameral Mydriatics (ICM) Injection enables injection of preservative free ophthalmic composition of 0.5 ml to 1 ml, without using any Preoperative to induce pupil dilation so that its diameter increases to a maximum of 4-6 mm within 30-90 seconds of injection given. This pupil dilation effect will hold up to30-120 minutes, which is safe time for phaco-emulsification and Intraocular lens (IOL) Implantation. The mean pupil diameter will be significantly greater than the than its normal dimension. No additional Mydriatics are needed up to the end of the surgery. The ophthalmic composition as embodied by the present invention can be directly injected in the Eye.

The said composition as embodied in the present invention comprises: at least three active ingredients. The said ingredients may be inclusive of, but not restricted to, Epinephrine Bitartrate, Atropine Bisulphate, Xylocaine Hydrochloride and additives like, antioxidant compounds, buffers and the like. The corresponding range of percentage(s) of Epinephrine Bitartrate in 1 ml is 0.07-0.21%, Atropine Bisulphate in 1 ml is 0.005-0.12%, and Xylocaine Hydrochloride in 1 ml is 92-99.9%. The three active ingredients are mixed in a predefined percentage range, and the composition is supplied in a sterile packaging containers such as vial/ampoule/prefilled syringes. It is very safe and effective for human subjects.

The composition as revealed in the present disclosure, is injected into the pupil, by employing a Intacameral Mydriatics (ICM).

### BRIEF DESCRIPTION OF DRAWINGS

The description of the present invention in conjunction with the drawings illustrating the present invention and in application(s) illustrative of the present invention, explain in detail, the working of the present invention. It will be amply clear to a person skilled in the art, that the application as disclosed in the drawings are merely illustrative of the scope of the present invention and not exhaustive of the scope of the present invention in its entirety. The advantages arising out of the illustrated application in conjunction with the enclosed drawings are merely indicative and are not to be construed to be restricting the scope of the present invention in any manner.

FIG. 1 shows the structure of the human Eye.

FIG. 2 shows the division of the Eye into the Anterior and Posterior Chambers (AC, PC).

FIG. 3 shows the variation in dimension of Iris under normal conditions, viz., widening of Iris under low-light conditions and contraction of Iris under bright light conditions.

FIG. 4 shows the conventional mechanical retraction device deployed for the widening of iris for surgical requirement.

FIG. 5 shows the J-clip which is used to hold the Iris, in the conventional mechanical retraction device deployed for the widening of iris for surgical requirement.

FIG. 6 shows the line diagram of the Iris, when widened by the mechanical retractor and post surgical profile of the iris.

FIG. 7 shows the line diagram of the Iris when injected by the composition as embodied by the present invention and its intact state after the surgery has been performed or the eye examination is completed.

FIG. 8 shows the diagram of the Intacameral Mydriatics (ICM) which is deployed in conjunction with the composition as embodied by the present invention.

### Detailed Description of Drawings

The result of modern-day advances in living conditions in general, and in the medical field in particular, is that, the average life-span of people has increased. However, with the progressive increase in life-span, there is also the problem of diagnosis and effective cure of Geriatric disorders, i.e., disorders that occur in the general population, due to Ageing, such as, Eye disorders for e.g., Cataract.

Cataract occurs predominantly with ageing and causes opacity of the Eye lens. As it is well-known to ophthalmic professionals and the general public alike, the human Eye (E) is an intricate structure, and a very crucial part of the human anatomy, and therefore, its well-being is very critical to the day-to-day smooth and independent functioning of people. Cataract is curable solely by surgery and, no medication exists as on date to cure Cataract. In such a scenario, it is highly important that, Cataract surgery be carried out under the best of conditions so that, the results of the surgery are conducive to the elderly patient(s) and not create any detriment in their day-to-day living thereafter. In order to carry out the Eye surgery inclusive of cataract efficiently, the pupil is required to be dilated to the required extent. The present invention is employable in such a context, however it is also employable for applications other than surgery such as Eye examination.

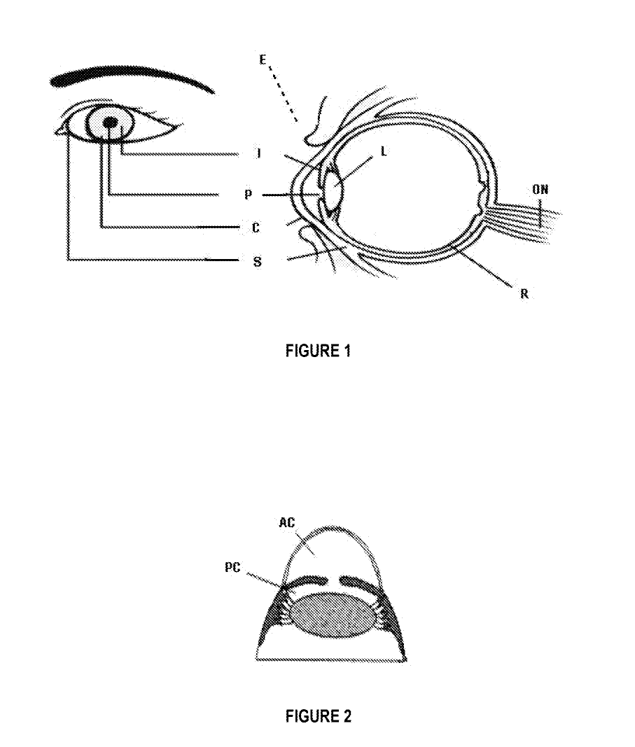

In this context, FIG. 1. shows the structure of the human Eye.

Structurally, the human Eye (E) is a slightly asymmetrical sphere inside a rounded area of the skull, and is filled with fluid. One component in the human Eye (E), is the Cornea (C). The Cornea (C) is the transparent front surface that curves over the Iris (I) and Pupil (P). The Eye (E) further comprises Lens (L) which refracts incoming light to focus it at the back of the Eye (E). The Cornea (C) is part of the Eyeball's wall and connects with the Sclera (S). The circular muscle behind the Cornea (C) controls the size of the Pupil (P) in its center, and determines quantity of light that can enter the Eye. The Pupil (P) itself is not a separate structure; but an opening in the Iris (I). Surrounding the Iris (I) is the Sclera (S). It is part of the total wall of the Eyeball and connects with the Cornea (C). The Eye further comprises, Anterior chamber (AC) and Posterior chamber (PC).

Further, the Anterior chamber (AC) and Posterior chamber (PC) of the Eye (E) are depicted in elaborate detail in the subsequent figure. In this context, FIG. 2 shows the division of the Eye into the Anterior and Posterior chambers (AC, PC).

From FIG. 2, it can be seen that, behind the Iris (I), is a cavity called the Anterior chamber (AC), filled with fluid, and behind that, is the Lens (L). The Anterior Chamber (AC) is the fluid-filled space inside the Eye between the iris and the Cornea's innermost surface, the endothelium. Aqueous humor is the fluid that fills the anterior chamber. After the cornea (C) admits light and bends it to a focus, the lens (L) bends it further. The combined refraction of cornea (C) and lens (L) focuses the light clearly on the retina (R) at the back of the Eye. The lens curvature is controlled by a circular muscle around it, called the ciliary muscle

Behind the lens (L) is a second fluid-filled area called the posterior chamber (PC). It makes up most of the Eyes (E) entire size. The retina (R) is the surface that borders it in a large curve extending almost to the ciliary muscle around the lens. Retinal cells are highly light-sensitive and receive the images in incoming light converting them to electrical energy. Near the center of the retina (R), is an opening, where the large optic nerve (ON) leaves the Eye.

In a network across the entire retina (R), are millions of tiny nerve fibers, each one connected to a single retinal cell. They pick up the electrical energy created by that cell from image information. They converge to form the optic nerve (ON) and leave the Eyeball, within a nerve sheath The optic nerve (ON) is connected to the brain's vision center, where the electrical energy it carries, is interpreted by the brain

Surgeries that are performed in the Anterior chamber of the Eye are exemplified as, LASIK, Iris Claw Lens Implant, Phakic Lens implant.

Surgeries that are performed in the Posterior chamber(PC) are exemplified as, Cataract surgery, Glaucoma. Retinal surgery, Vitreous surgery and implant of other devices in the posterior chamber of the Eye.

A person skilled in the art will understand that, the example(s) provided herein and elsewhere in the present disclosure, are merely provided for the purpose(s) of illustration and the above-mentioned example(s), are merely for the purpose(s) of understanding.

The present invention is embodied by an Ophthalmic composition, that is deployed for the purpose of dilating the Pupil (P), for carrying out accurate and efficient surgeries in the Posterior chamber of the Eye such as Cataract surgery and Eye examination.

Persons skilled in the art, for instance, Ophthalmic professional(s), who are knowledgeable in the intricate structure and efficient functioning of the human Eye(E), will be aware that, Iris (I) is the component of the human Eye, comprising of the Pupil (P), which enables the control of light, that is incident on the Retina (R), said Retina (R) comprising, millions of tiny nerve fibers, each one connected to a single Retinal cell, which picks up the electrical energy created by that cell, from image information.

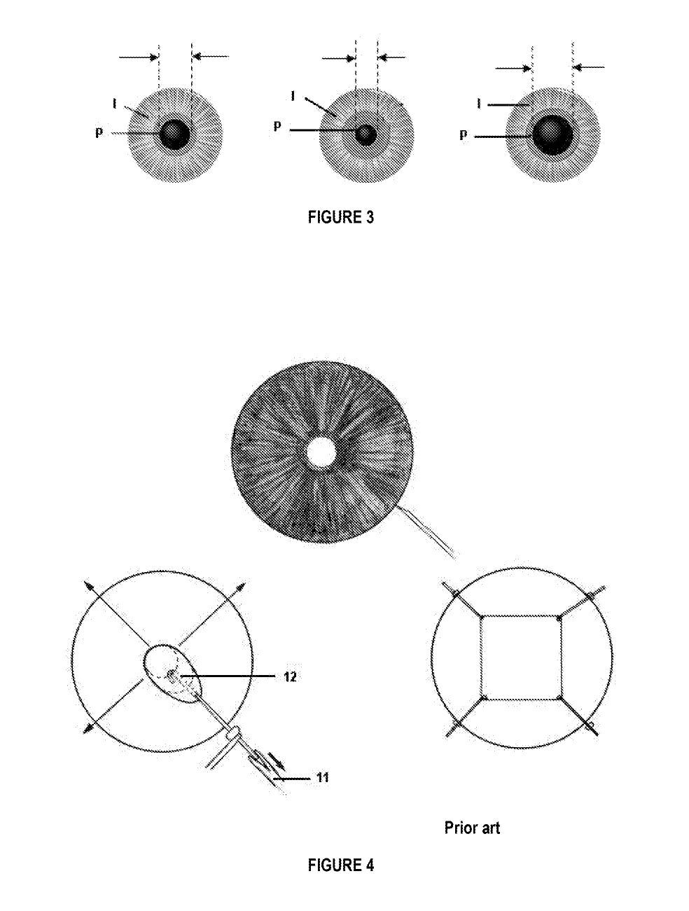

In this context, Iris (I) can be described as the component, which controls the amount of light, incident on the sensitive Retinal cells. This functioning of Iris, is actually a protection for the sensitive Retinal cells, which have the likelihood of damage under excessive light. The Iris (I), and its dimensional variation, in accordance with the light availability, is shown in elaborate detail, in conjunction with FIG. 3.

FIG. 3 shows the variation in the dimension of Iris (I) under normal conditions, viz.., widening of Iris under low—light conditions and contraction of Iris under bright—light conditions.

Further, the Iris is a contractile structure, consisting mainly of smooth muscle, surrounding the Pupil (P). Light enters the Eye (E) through the Pupil (P), and the Iris regulates the amount of light by controlling the size of the Pupil. The Pupil is a hole-type structure located in the center of the Iris (I) of the Eye (E) that allows light to enter the Retina (R). It appears black, because, most of the light entering the Pupil (P) is absorbed by the tissues inside the Eye. In humans, the Pupil is round. The image of the Pupil as seen from outside the Eye is the entrance Pupil, which does not exactly correspond to the location and size of the physical Pupil because it is magnified by the Cornea (C). When the Sphincter Pupillae contract, the Iris decreases or constricts the size of the Pupil. The dilator Pupillae, innervated by sympathetic nerves from the superior Cervical Ganglion, cause the Pupil to dilate when they contract. These muscles are sometimes referred to as intrinsic Eye muscles. The sensory pathway (rod or cone, bipolar, ganglion) is linked with its counterpart in the other eye by a partial crossover of fibers of each Eye.

When bright light is shone on the Eye, light sensitive cells in the Retina (R), including rod and cone photoreceptors and melanopsin ganglion cells, will send signals to the oculomotor nerve, specifically, the parasympathetic part coming from the Edinger-Westphal nucleus, which terminates on the circular Iris (I) Sphincter Muscle. When this Muscle contracts, it reduces the size of the Pupil (P). The Pupil (P) gets wider in the dark but narrower in light. When narrow, the diameter is 3 to 5 mm. In the dark it will be the same at first, but will approach the maximum diameter for a wide Pupil 4 to 8 mm. In any human age group there is however considerable variation in maximal Pupil (P) size. For example, at the peak age of 15, the dark-adapted pupil can vary from 4 mm to 9 mm with different individuals. After 25 years of age, the average pupil (P) size decreases, though not at a steady rate. At this stage, the pupils do not remain completely still, therefore may lead to oscillation, which may intensify and become known as hippus. In addition to age, other factors like heredity, country, determine the Eye colour as well as diameter of the pupil and hence small pupil is a relative term. When only one Eye is stimulated, both Eyes contract equally. The constrictions of the Pupil and near vision are closely tied. In bright light, the pupils constrict to prevent aberrations of light rays and thus attain their expected acuity; in the dark this is not necessary, so it is chiefly concerned with admitting sufficient light into the Eye.

It will be amply clear to person(s) skilled in the art, that, the widening of Iris (I) under low-light conditions, and contraction under bright—light conditions, is in line with the normal functioning of the Iris (I), without manual intervention, and is therefore, voluntary in nature. However, prior to starting the surgery, there is a requirement to widen the Pupil (P), within the Iris (I). Under such conditions, in its normal mode, the Iris (I) would have contracted since, the microscope illumination deployed during surgery is very bright. But for the effective carrying out of the surgery, the surgeon has the requirement for the pupil to be widened to the maximum. Widening of the pupil may be required for non surgical applications like Eye examination for instance. Since such widening is not possible naturally, without manual intervention, the present invention embodied as an Ophthalmic Composition for pupil dilation in the context of Eye examination and surgeries in the posterior chamber of the Eye such as Cataract Surgery, is required.

Even though, an Ophthalmic Composition for Pupil dilation in the context of Eye surgery in general, and Cataract Surgery in particular, has been employed in the context of the present invention, however, the dilation of pupil has been achieved in the prior art by means of an instrument which deploys a mechanical means of holding iris by J shaped clips. Such an instrument and its method of deployment, have been described in detail, in the subsequent figure.

In this context, FIG. 4 shows a conventional mechanical retraction device (RD)deployed for the widening of Iris (I), for surgical requirement. FIG. 4 shows an Iris Retractor (RD) for use in operating on the Eye (E) by means of which, the Iris (I) is drawn outwards for widening the Pupil (P) and is held at the outer edge, which is essentially formed by the transition from the Cornea (C) to the Sclera (S). The Iris Retractor (RD)essentially comprises a suspended part having a hook portion (12) and a guide part and on which a platelet-like clamping part (11) is displaceable relative to the hook portion. The FIG. 4 shows Eye with the Iris region retracted by means of the iris retractor. The retractor is held by the clamping part arranged displaceably on the suspended part and engaging at the transition of the Eye. The clamping part is made from corresponding flexible material, so that, the side facing the transition can adapt to the arcuate transition. It will be very apparent to a person skilled in the art that, the retractor for use in operating on the Eye as described above, is merely for the purposes of illustration only.

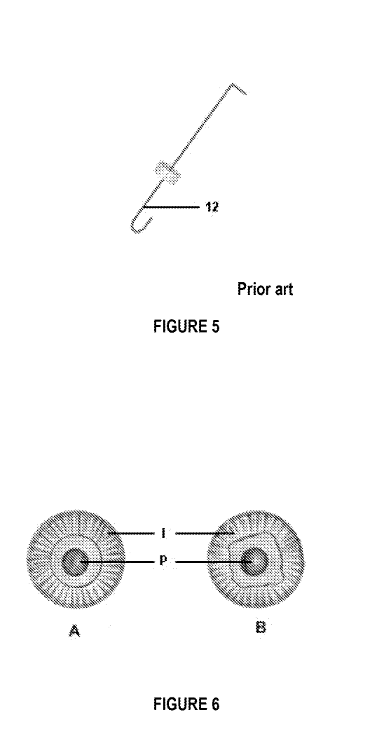

The important portion of the Iris Retractor device is the hook portion. (12).It is a medically known fact that, the hook portion is inserted into the Iris and leaves deformities in the Iris, due to its insertion. The said deformities are expected to heal with time, however, the healing may not be complete or maybe, a partial healing, given the fragile nature of the Iris and its delicate texture. The hook portion has been shown in FIG. 5. If the said healing is not complete, it means that, the functioning of the Iris and the Retina may be affected. Such an effect may result in long term vision impairment. Such an injury to the Iris is termed as “Iris laceration” by person(s) skilled in the art. If on the other hand, the hook portion is not inserted properly, it may result in the surgery not having the desired result. The said fact can be appreciated on observing the line diagram which shows the configuration of the Iris (6A) before surgery and configuration of Iris (6B) after surgery is performed. FIG. 6(A,B) shows such a line diagram. It will be duly appreciated by persons skilled in the art that, the line diagram is only illustrative of the laceration that is likely to be caused to the Iris, by deployment of the said Iris Retractor.

On the other hand, if the Opthalmic composition as embodied in the present invention, is deployed, via an Intacameral Mydriatics (ICM) Injection which enables injection of preservative free composition of 0.5 ml to 1 ml, without using any Preoperative to induce pupil dilation maximum 4-6 mm within 30-90 seconds of injection given directly into the Eye. This pupil dilation effect will hold up to 30-120 minutes, which is safe time for phaco-emulsification and Intraocular lens (IOL) Implantation. The mean pupil diameter will be significantly greater than the normal pupil diameter. No additional Mydriatics are needed up to the end of the surgery..

The composition as embodied in the present invention comprises: at least three active ingredients. The said ingredients may be inclusive of, but not restricted to, Epinephrine Bitartrate, Atropine Bisulphate, Xylocaine Hydrochloride and additives like, antioxidant compounds, buffers and the like.

The corresponding range of percentage(s) of Epinephrine Bitartrate in 1 ml is 0.07-0.21%, Atropine Bisulphate in 1 ml is 0.005-0.12%, and Xylocaine Hydrochloride in 1 ml is 92-99.9%. The three active ingredients are mixed in a defined percentage, and the composition is supplied in a packaging container inclusive of sterile package, such as, vial or ampoule or prefilled syringes. It is very safe and effective for human subjects.

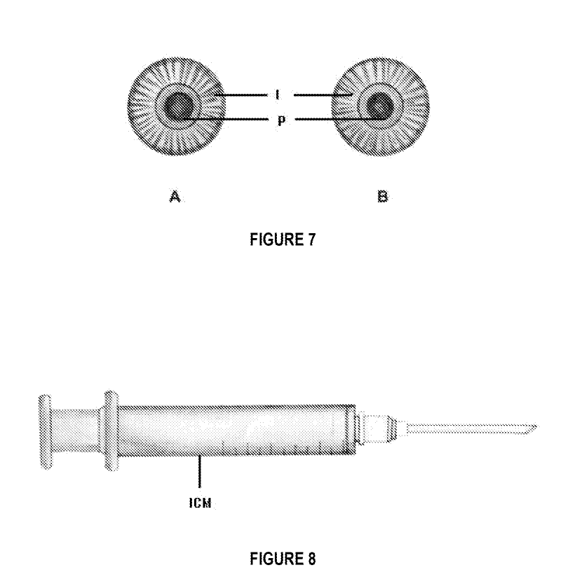

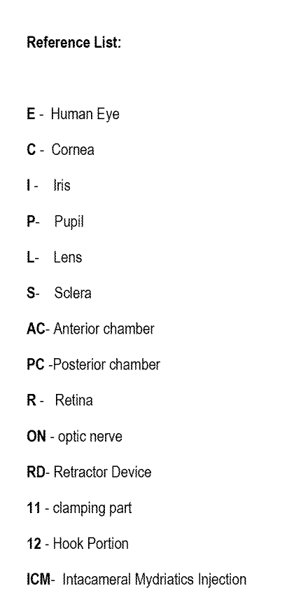

Therefore, since no hook portion is deployed, Iris laceration does not happen, and therefore the profile of the Iris is intact. It is seen from FIG. 7 (A,B) that the profile of the Iris (7A) prior to surgery is the same as profile of Iris (7B) after surgery is completed. It is very clear that the profile of Iris is intact unlike the laceration as seen in FIG. 6B. The said profile and dimension of the Iris is merely illustrative. It is known to ophthalmic professions that the size of the pupil is dependant on age and genetic factors. In this context, the size of the pupil may be smaller than the normal pupil. Generally pupil(s) of dimension 2.5 mm diameter are termed small pupil. The ophthalmic composition as exemplified by the present invention is deployable for such small pupil(s) also. Further it is amply clear to persons skilled in the art that not all pupils have the same nature and some pupils do not dilate as quickly as normal pupils and ophthalmic professionals term such pupils as “sluggish pupils” Such pupils are also dilated quickly using Ophthalmic Compositions as exemplified in the present invention and do not suffer from any lacerations in the Iris post surgery. When the Opthalmic composition as embodied in the present invention, is deployed, an Intacameral Mydriatics (ICM) Injection enables injection of preservative free composition. An injection of this type is illustrated in FIG. 8. It will however be understood by a person skilled in the art that, the injection as depicted in the illustration is merely indicative and is in no way restrictive to the type of the injection that may be deployed. Any injection of similar functionality may also be deployed and such deployment falls within the scope and purview of the present invention.

### ADVANTAGES OF THE INVENTION

The present invention discloses an Ophthalmic Composition, which is deployed for Eye examination and surgeries carried out in the Posterior chamber of the Eye such as Cataract surgery. The advantage(s) arising out of the deployment of the present invention, in the said application, are numerous, however, it would be clear to a person skilled in the art, that, the advantage(s) arising out of the present invention have been enclosed merely, as an illustration serving as an indication, pertaining to the nature of advantage(s), arising as a result of the deployment of the present invention. Further still, it would be amply clear to a person skilled in the art that, the allied advantage(s) arising as a result of the present invention, and the secondary advantage(s) arising as a result of the below-mentioned primary advantage(s), are to be construed to be within the scope and ambit of the present disclosure.

In the above-mentioned context, an advantage of the present invention is, it provides an ophthalmic composition, which enables dilating the pupil during the Eye examination and surgeries carried out in the Posterior chamber of the Eye such as cataract surgery, especially for pupil(s) relatively smaller in dimension than normal pupil.

The ophthalmic composition as exemplified by the present invention enables dilating even “sluggish pupils” and reduces the time of surgery.

It also reduces the effort of the personnel involved since in conventional surgeries, clinical examination of the pupil has to be performed to determine if the required pupil dilation is present prior to performing the surgery. In the present invention, such clinical examination is not required since the patient can undergo the surgery as soon as the ophthalmic composition has been administered.

Yet another advantage of the present invention is, it provides an ophthalmic composition, which enables dilating the pupil during the Eye examination and surgeries carried out in the Posterior chamber of the Eye such as cataract surgery, in a manner that the dilated pupil is capable of being restored to its original dimensions, after the surgery has been completed.

Still another advantage of the present invention is, to provide an ophthalmic composition, using which, it is possible to dilate the pupil during the Eye examination and surgeries carried out in the Posterior chamber of the Eye such as cataract surgery, in a manner so that, the composition is nontoxic to the Eye.

Further still, an advantage of the present invention is to provide an ophthalmic composition, in which, it is possible to dilate the pupil during Eye examination and surgeries carried out in the Posterior chamber of the Eye such as cataract surgery, in a manner such that, the composition retains the pupil in the same dimension, during the duration in which the surgery is performed.

A further advantage of the present invention is, to provide an ophthalmic composition, using which, it is possible to dilate the pupil without affecting the position or condition of the surrounding parts of the Eye, the Eye being an intricate and delicate organ, wherein, any minor change in positioning and/or condition of the surrounding parts of the Eye is capable of affecting the outcome of the surgery.

A further advantage of the present invention is, to provide an ophthalmic composition, using which, it is possible to dilate the pupil without causing any laceration to the pupil resulting in deformities, due to which, there may be a long term and permanent vision impairment.

Further still, an advantage of the present invention is, to provide an ophthalmic composition, in which, it is possible to dilate the pupil during Eye examination and surgeries carried out in the Posterior chamber of the Eye such as cataract surgery, without the requirement of the person effecting the pupil dilatory composition to be a very highly skilled ophthalmic professional for instance, a person in the league of an Eye surgeon. A person skilled in the art is aware that, deployment of Iris Retractors requires skilled ophthalmic personnel in the league of Eye surgeon to deploy the Iris Retractor.

Further still, an advantage of the present invention is to provide an ophthalmic composition, using which, it is possible to dilate the pupil during Eye examination and surgeries carried out in the Posterior chamber of the Eye such as cataract surgery, without the requirement of a very costly instrument, since Iris Retractors are very expensive in comparison to Chemical Compositions.

Further still, an advantage of the present invention is to provide an ophthalmic composition, using which, the Iris returns to its original configuration without any ruptures causing deformities in its structure, after the surgery is completed.

We claim:

1. An ophthalmic composition contained in a packaging container, for enabling dilation of pupil(s) during ophthalmic application(s), said dilation of pupil(s) enabled by deployment of an injecting instrumentation, said composition comprising:
(a) Epinephrine Bitartrate, said Epinephrine Bitartrate being one of the active ingredients and forming part of the said composition in predefined percentage range in a predefined volume;
(b) Atropine Bisulphate, said Atropine Bisulphate being one of the active ingredients and forming part of the said composition in predefined percentage range in a predefined volume;
(c) Xylocaine Hydrochloride, said Xylocaine Hydrochloride being one of the active ingredients and forming part of the said composition in predefined percentage range in a predefined volume;
(d) and additives.

2. The Ophthalmic composition for enabling dilation of pupil(s) during ophthalmic application(s), as claimed in claim 1, wherein, pupils is indicative of pupils which are relatively smaller in dimension than the normal pupils.

3. The Ophthalmic composition for enabling dilation of pupil(s) during ophthalmic application(s), as claimed in claim 1, wherein, ophthalmic application(s) is inclusive of Eye examination, surgeries carried out in the posterior chamber of the Eye like cataract surgery.

4. The Ophthalmic composition for enabling dilation of pupil(s) during ophthalmic application(s), as claimed in claim 1, wherein, packaging container is inclusive of a sterile package such as, vial, prefilled syringes or ampoule.

5. The Ophthalmic composition for enabling dilation of pupil(s) during ophthalmic application(s), as claimed in claim 1, wherein, injecting instrumentation is inclusive of Intacameral Mydriatics (ICM) Injection and the like.

6. The Ophthalmic composition for enabling dilation of pupil(s) during ophthalmic application(s), as claimed in claim 1, wherein, predefined percentage range of Epinephrine Bitartrate, said Epinephrine Bitartrate being one of the active ingredients, 0.07-0.21%.

7. The Ophthalmic composition for enabling dilation of pupil(s) during ophthalmic application(s), as claimed in claim 1, wherein, predefined percentage range of Atropine Bisulphate said Atropine Bisulphate being one of the active ingredients, is 0.005-0.12%.

8. The Ophthalmic composition for enabling dilation of pupil(s) during ophthalmic application(s), as claimed in claim 1, wherein, predefined percentage range of Xylocaine Hydrochloride said Xylocaine Hydrochloride being one of the active ingredients is 92-99.9%.

9. The Ophthalmic composition for enabling dilation of pupil(s) during ophthalmic application(s), as claimed in claim 1, wherein, predefined volume is 1 ml.

10. The Ophthalmic composition for enabling dilation of pupil(s) during ophthalmic applications, as claimed in claim 1, wherein, additives are inclusive of antioxidant compounds, buffers and the like.
